(19) United States
(12) Patent Application Publication
(10) Pub. No.: US 2006/0069069 A1
(43) Pub. Date: Mar. 30, 2006

(54) Methods and compositions for the administration of calcium chelators, bisphosponates and/or citrate compounds and their pharmaceutical uses

(75) Inventors: E. Olavi Kajander, Kuopio (FI); K.M. Aho, Kuopio (FI); Neva Ciftcioglu, (US); B. Millican, (US)

Correspondence Address: HOGAN & HARTSON LLP;IP GROUP, COLUMBIA SQUARE, 555 THIRTEENTH STREET, N.W., WASHINGTON, DC, 20004, US

(73) Assignee: Nanobac Pharmaceuticals, Inc.

(21) Appl. No.: 11/180,921

(22) Filed: Jul. 14, 2005

Related U.S. Application Data
(60) Provisional application No. 60/587,869, filed on Jul. 15, 2004.

(51) Int. Cl. A61K 31/675; A61K 31/195; A61K 31/19

(52) U.S. Cl. 514/89; 514/102; 514/566; 514/574

(57) ABSTRACT

A composition is provided which contains calcium chelators, bisphosphonates, and/or citrate compounds and which may be used for treating and or reducing pathological calcifications, heavy metal poisoning, the growth of Nanobacterium Calcifying Nano-Particles and calcification-induced diseases in humans and animals. The method includes administering a therapeutic composition of calcium chelators, bisphosphonates, and/or citrate compounds which effectively inhibit or treat the development of calcifications in vivo. Typically, the administered composition includes about 0.1-10:1 parts by weight of calcium chelators, bisphosphonates, and/or citrate compounds.

### CROSS REFERENCE TO RELATED APPLICATIONS

This application claims priority from U.S. Provisional Patent Application Ser. No. 60/587,869, filed Jul. 15, 2004, the disclosure of which is hereby incorporated by reference in its entirety.

### BACKGROUND OF THE INVENTION

1. Field of the Invention

The invention relates to therapeutic compositions and methods for the administration of calcium chelators, bisphosphonates and/or citrate compounds and more particularly to therapeutic compositions and methods for treating and/or preventing pathological and other calcifications by administering preparations of calcium chelators bisphosphonates and/or citrate compounds either separately or in concert.

2. Discussion of the Art

The formation of discrete and organized inorganic crystalline structures within macromolecular extra cellular matrices is a widespread biological phenomenon generally referred to as biomineralization. One example of biomineralization is the formation of calcium phosphate. When calcium phosphate is deposited in tissue, it is known as calcification. Mammalian bone and dental enamel are examples of calcification.

Pathological calcification is not the healthy process that builds bones and teeth, but instead it is found in disease. Pathological calcification is found in a variety of diseases. Although the cause of pathological calcification remains unknown, it has been observed that the condition is often associated with the presence of a very small, mineral-associated bacteria-like self-propagating calcium-phosphate macromolecular complexes/forms termed Nanobacteria (Nanobacterium sanguineum) or Calcifying Nano-Particles which are known self-propagating calcium-phosphate macromolecular complexes/forms termed Nanobacteria (Nanobacterium sanguineum) or Calcifying Nano-Particles which are known for their ability to create calcium phosphate coated vesicles or nano-particles that multiply in blood and in cell culture medium like living cells. Nanobacteria/Calcifying Nano-Particles (“NB/CNP”) are approximately 20-200 nanometers in size and are currently the smallest known self-replicating particles or bacteria.

NB/CNP are causal to calcification by building calcium-phosphate mineral deposits or “envelopes” around each cell or particle. NB/CNP secrete a calcific biofilm around itself that protects the particle and allows for multiple NB/CNP to connect, collaborate and apparently form together as a unit or colony. This calcific biofilm also allows the NB/CNP to expand, contract and move.

This biofilm-phase appears to be present when NB/CNP are chemically attacked, physiologically stressed, environmentally attacked or when they are working together or multiplying. During the biofilm-phase, when NB/CNP secretes the calcium-phosphate mineral, the NB/CNP is most harmful. The calcified plaques caused by NB/CNP are formed because the calcium phosphate mineral agglomerates into particles forming an exposed biofilm that activates a thrombic cascade. The calcific biofilm that is secreted by the NB/CNP includes a potent endotoxin and causes inflammation and swelling, causing the surrounding tissue to respond by releasing cytokines, interleukins, leukocytes, mast cells, collagenase, matrix metalloproteinases and other immune-responsive reactions. Many of the medical markers of inflammation, such as C-reactive protein, are found to be elevated in response to the endotoxin in the NB/CNP biofilm.

NB/CNP may also form the calcific biofilms and propagate under blood/serum conditions. NB/CNP are the only calcium-phosphate mineral containing particles isolated from human and cow blood that are cytotoxic in vitro and in vivo. Human and bovine NB/CNP grow similarly, share the same surface antigens and various other features. They both produce biomineralization. Most biologicals and vaccines are made using fetal bovine serum.

The ability to study NB/CNP has been difficult. Many of the chemicals used to stain cell walls or other components of traditional bacterial fail to bind to NB/CNP. Also, NB/CNP do not thrive on agar, the medium used to grow most bacteria. As such, the ability to culture NB/CNP and to develop NB/CNP antibodies has been difficult. Commonly assigned U.S. Pat. No. 5,135,851, incorporated herein by reference, describes a culture method for NB/CNP.

NB/CNP cannot be grown on standard media for bacteria, and thus they escape detection when using standard culture methods. The detection of the extremely small unidentified particles is hampered by their size, which, e.g., in commercial cell culture isolates, is smaller than 0.5 micro-meters. Thus, their detection via light microscopy is possible only with the best microscopes having maximum resolution. Tissue culture laboratories are seldom equipped with such microscopes. Further, these nano-particles are difficult to collect since centrifugation is difficult. Still further, whereas NB/CNP do adhere to glass, it is difficult to fix NB/CNP to glass slides for conventional microbial analysis and they cannot be stained with common bacteriological stains. The growth requirements of various isolates of NB/CNP are quite similar. The growth requirements can be met using standard tissue culture media. This is likely because these NB/CNP are adapted for living inside the mammalian body.

The ability to detect NB/CNP is also very difficult. As noted above, NB/CNP are extremely small, approximately 1/1,000 the size of most other bacteria. NB/CNP show very slow replication, doubling their population approximately every 3 days Most bacteria reproduce in minutes or in hours. Also, NB/CNP are pleomorphic in that they have varying forms or shapes during their life cycle and can change appearance and form during growth and development. Because of their extremely small size, slow growth rate, and pleomorphism, NB/CNP often avoid detection. Commonly assigned U.S. Pat. No. 5,135,851, incorporated herein by reference, describes a method for detecting NB/CNP.

NB/CNP have been observed to be associated with pathological calcification found in a wide range of diseases. NB/CNP induced pathological calcification is implicated to be either associated with or an instrumental component of most degenerative disease processes. These include heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Keratopathy, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Pyoderma gangrenosum, Dermatomyositis, eccrine sweat duct calcification, trichoepithelioma, pilomatrixoma, necrobiosis lipoidica, Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

In addition to the pathological calcification associated with NB/CNP; trauma, stress, surgery and/or biological implants are associated with pathological calcification. In particular, biological implants (e.g. prosthesis) are vulnerable to undesired calcification. Bioprosthetic devises in which calcification is a serious problem include, but are not limited to, heart bioprotheses, homografts/allographs (human cadaver), autografts, mechanical bioprotheses and implants such as urinary and heart stents, particularly those made using polyetherurethaneurea and polyetherurethane, silicone implants (including breast implants) and other synthetic materials.

NB/CNP have been found to be a contaminant on previously-assumed-to-be sterile medical products, such as tissue, blood and bovine serum. NB/CNP are extremeophiles and exhibit a greater resistance to extreme environments than most bacteria to destruction. NB/CNP cannot be killed using most antibiotics, such as Penicillin, Cephalosporins, or Macrolides. NB/CNP are also tolerant to very high heat, freezing, dehydration and Gamma Irradiation.

Chelation therapy has been proposed to treat and/or prevent existing atherosclerosis caused by pathological calicification. Chelation therapy is administering ethylenediamine tetraacetic acid (EDTA), a man-made amino acid, into the veins. EDTA has often been used in cases of heavy metal poisoning (lead or mercury) because it can bind these metals, creating a compound that can be excreted in the urine. Besides binding heavy metals, EDTA also chelates calcium. This has led to the speculation that EDTA could remove calcium deposits from buildup or calcific lesions in the arteries.

At present, chelation therapy is normally administered intravenously to a patient who must remain relatively immobile. It is believed that oral ingestion of EDTA is impractical because stomach acids destroy its effectiveness. A single intravenous chelation treatment usually lasts about four hours and is generally administered three times a week for about three months. Patients often are advised to continue preventative treatment once or twice a month, over a two-year period. Such frequent immobilization inconveniences the patient, and requires considerably large and dedicated floor space at the administration facility.

Because NB/CNP and prostheses cause and/or increase pathological calcification, and because pathological calcification is increasingly linked with numerous diseases, it would be advantageous to provide unique alternatives to the arduous intravenous chelation therapy, which can be used to inhibit and/or prevent calcification in vivo and to inhibit and/or prevent the growth of NB/CNP in vivo.

### SUMMARY OF THE INVENTION

The present invention provides a therapeutic composition for reducing calcifications in vivo and a method for reducing the growth of Nanobacteria/Calcifying Nano-Particles in humans and animals by administering the composition of the present invention. The composition of the present invention comprises a mixture of calcium chelators, bisphosphonates and/or citrate compounds. The composition further comprises from 0% to about 65% by weight of pharmaceutically acceptable formulation aids, such as diluents, stabilizers, binders, buffers, lubricants, coating agents, preservatives, emulsifiers and suspension agents.

In accordance with embodiments of the invention, the calcium chelators may include one or more of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof.

In accordance with the invention, the bisphosphonates may include one or more of alendronate, clodronate, ibandronate, incadronate, neridronate, palmidronate, risedronate, tiludronate, zoledronate, etidronate, oxidronate, and pharmaceutically acceptable salts thereof.

In accordance with the invention, the citrate compounds may include one or more of citrate, including sodium and potassium salts, magnesium citrate, phosphocitrate and other complexes of citrate or other organic and inorganic derivatives thereof.

In still another aspect, the present invention relates to a method for treating or preventing the development of calcifications in vivo, heavy metal poisoning, Nanobacteria Calcifying Nano-Particles and calcification associated diseases which comprises administering a pharmaceutically effective amount of a composition comprising calcium chelators, bisphosphonates and/or citrate compounds to a mammal, e.g., a human.

In yet another aspect, the present invention relates to a method of using a composition comprising calcium chelators, bisphosphonates and/or citrate compounds which comprises administering said composition to reduce and/or prevent calcification related diseases, such as heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Keratopathy, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Pyoderma gangrenosum, Dermatomyositis, eccrine sweat duct calcification, trichoepithelioma, pilomatrixoma, necrobiosis lipoidica, Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease in an individual in need thereof. The daily dosage is established between 0.1 to 3,000 mg of calcium chelators, bisphosphonates and/or citrate compounds (preferably 100 to 1,500 mg) per day and is intended for ingestion in any type or form of foodstuff, capsule, tablet or liquid form.

In still yet another aspect, the present invention relates to a method of using a composition comprising bisphosphonates and/or citrate compounds in combination for treating nephrolithiasis or “renal stone disease” wherein hardened mineral deposits form in the kidneys that originate as microscopic particles or crystals and develop into stones over time. Approximately 85% of all Kidney stones are comprised of calcium. These stones are usually a combination of calcium and oxalate. A number of factors can cause high concentrations of these substances in the urine.

Therefore, one aspect of this invention is to provide a composition comprising at least one of calcium chelators, bisphosphonates, and or citrate compounds.

Another aspect of this invention is to provide a composition comprising at least one of calcium chelators, bisphosphonates, and or citrate compounds for the treatment or prevention of the development of calcifications in vivo.

Yet another aspect of this invention is to administer a pharmaceutically or therapeutically effective amount of a composition comprising at least one of calcium chelators, bisphosphonates, and/or citrate compounds to a human or mammal.

Still yet another aspect of this invention is to administer a pharmaceutically or therapeutically effective amount of a composition comprising at least one of bisphosphonates and/or citrate compounds for the treatment of kidney stones.

### DETAILED DESCRIPTION OF THE PREFERRED EMBODIMENTS

This application incorporates by reference, in its entirety, co-pending, commonly assigned United States non-provisional patent application entitled Methods and Compositions for the Treatment of Diseases Characterized by Pathological Calcification, filed on Jul. 15, 2004, attorney docket number 19772.0004.

The invention provides for therapeutic compositions and methods for treating and/or preventing pathological calcifications, heavy metal poisoning, and the growth of Nanobacterium/Calcifying Nano-Particles (“NB/CNP”)by administering preparations of calcium chelators, bisphosphonates, and/or citrate compounds, and for treating and/or preventing calcification-associated diseases including, but not limited to, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Keratopathy, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Pyoderma gangrenosum, Dermatomyositis, eccrine sweat duct calcification, trichoepithelioma, pilomatrixoma, necrobiosis lipoidica, Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

The methods involve administering to a patient a therapeutically effective amount of calcium chelators, bisphosphonates, and/or citrate compounds. In view of the foregoing, the methods of this invention are particularly applicable where the patient is at risk for or has NB/CNP infection, Coronary Artery Disease, and/or where the patient will have or has had surgery and/or biological implants. 031 The composition of the present invention comprises a formulation of at least one of calcium chelators, bisphosphonates and/or citrate compounds as the primary therapeutic agent(s) to be administered for the purpose of reducing and/or preventing pathological calcifications, heavy metal poisoning, NB/CNP, and preventing calcification-associated diseases including, but not limited to, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Keratopathy, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Pyoderma gangrenosum, Dermatomyositis, eccrine sweat duct calcification, trichoepithelioma, pilomatrixoma, necrobiosis lipoidica, Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease in an individual in need thereof.

As discussed above, NB/CNP produce biomineralization by forming a calcific biofilm. The mineral coating constitutes a part of the cell wall essential for survival strategy of the organism. In NB/CNP, the mineral is basic calcium phosphate typically in the form of carbonate apatite. NB/CNP use the calcific biofilm to catalyze its metabolic and physiological processes and to provide it with structural support. Thus, a calcium chelator that is targeted to the apatite may be useful for the treatment of pathological calcifications, NB/CNP, and calcification-associated diseases.

The use of bisphosphonates and/or citrate compounds are used also as the primary therapeutic agent(s) to be administered for the purpose for treating nephrolithiasis or “renal stone disease” wherein hardened mineral deposits form in the kidneys that originate as microscopic particles or crystals and develop into stones over time. Approximately 85% of all Kidney stones are comprised of calcium. These stones are usually a combination of calcium and oxalate. A number of factors can cause high concentrations of these substances in the urine.

The calcium chelators currently available for use in the present invention and the associated daily recommended dosage include one or more of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof. The dose of these medicines will be variable for different patients.

Similarly, bisphosphonates, may be useful to target and destroy the calcific biofilm. Bisphosphonates are characterized pharmacologically by their ability to inhibit bone resorption, whereas, pharmacokinetically, they are classified by their similarity in absorption, distribution, and elimination. Bisphosphonates have a P—C—P bond instead of the P—O—P bond of inorganic pyrophosphate that makes them resistant to enzymatic degradation and gives them a high affinity for hydroxyapatite. They are potent blockers of osteoclasic bone resorption and have been successfully used to treat metabolic bone diseases that involve increased bone resorption. Bisphosphonates also inhibit bone mineralization and soft tissue calcification.

Bisphosphonates suitable for use in the present invention include, but are not limited to, alendronate, clodronate, ibandronate, incadronate, neridronate, palmidronate, risedronate, tiludronate, zoledronate, etidronate, oxidronate, and pharmaceutically acceptable salts thereof. It is possible to synthesize a variety of bisphosphonates by substituting the hydrogen on the carbon atom. The dose of these medicines will be variable for different patients.

In accordance with the invention, the citrate compounds may include one or more of citrate including sodium and potassium salts, magnesium citrate, phosphocitrate and other complexes of citrate, or other organic and inorganic derivatives thereof. The dose of these medicines will be variable for different patients.

The formulations of the present invention comprise compositions made by combining calcium chelators, bisphosphonates, and/or citrate compounds. Such compositions can comprise calcium chelators, bisphosphonates, and/or citrate compounds in a quantitative ratio from about 100:1 to about 00.1:1 by weight, to from about 10:1 to about 0.10:1 by weight. Compositions of the present invention may further contain 1:1 weight ratios of calcium chelators, bisphosphonates, and/or citrate compounds.

Total doses of the calcium chelators, according embodiments of the present invention may range from 0.1 to 3,000 mg/day, to 10 to 2,000 mg/day to 100 to 1,500 mg/day.

Total doses of the bisphosphonates depend heavily upon the type of bisphosphonate used. For example, alendronate, an aminobisphosphonate, is approximately 700-fold more potent than etidronate for the prevention of bone resorption, both in vitro and in vivo.

With respect to the calcium chelators, bisphosphonates and/or citrate compounds, the dose may be in the range of 0.1-3,000 mg/day. In another embodiment the dose may be in the range of 10-2,000 mg/day, and in another embodiment the dose may be in the range of 100-1,500 mg/day.

The compositions of the present invention can be taken in amounts sufficient to provide the desired dosages discussed above.

Formulations:

The pharmaceutical formulations of the present invention can contain as active ingredients from about 0.5 to about 95.0% wt of calcium chelators, bisphosphonates, and/or citrate compounds. This dosage is obtained by mixing the composition of the present invention with different excipients such as agglutinants, disintegrators, lubricants, sliders or just fillers. These excipients include but are not limited to lactose, corn starch, saccharose, magnesium stearate, microcrystalline cellulose, sodium croscarmellose gelatin, cellulose acetophtalate, titanium dioxide, silicon dioxide, precipitated silicates, fumed silicates, special talc for tablets and polyethylene glycol.

The pharmaceutical composition of the present invention may be administered to humans and animals. The daily dosage of this composition to be used for inhibiting and/or preventing pathological calcification, heavy metal poisoning, NB/CNP, and calcification-induced diseases including, but not limited to, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Keratopathy, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Pyoderma gangrenosum, Dermatomyositis, eccrine sweat duct calcification, trichoepithelioma, pilomatrixoma, necrobiosis lipoidica, Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease is established between 0.1 to 3,000 mg/day for the calcium chelator/bisphosphonate/citrate constituent, depending on which calcium chelators, bisphosphonates, and/or citrate compounds are present.

The therapeutic composition of the present invention may be packaged in any convenient, appropriate packaging.

As will be appreciated by one knowledgeable in the art, the therapeutic composition of the present invention may be combined or used in combination with other treatments.

1. Oral Administration:

The compositions of the invention may be in forms suitable for oral use (for example as tablets, solutions for sublingual administration, lozenges, hard or soft capsules, aqueous or oily suspensions, emulsions, dispersible powders or granules, syrups or elixirs).

Suitable pharmaceutically-acceptable excipients for a tablet formulation include, for example, inert diluents such as lactose, sodium carbonate, sodium sulfate, granulating and disintegrating agents such as corn starch or algenic acid; binding agents such as starch, PVP, CMC; lubricating agents such as stearate, stearic acid, fumed silica or talc; preservative agents such as ethyl or propyl p-hydroxybenzoate, sodium benzoate, potassium benzoate and anti-oxidants, such as ascorbic acid or tocopherol acetate. Tablet formulations may be uncoated or coated either to modify their disintegration and the subsequent absorption of the active ingredient within the gastrointestinal tract, or to improve their stability and/or appearance, via liposomal and or nanoemulsion techniques, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucralose, stevia, sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The amount of the active ingredients comprising the composition of this invention that is combined with one or more excipients to produce a single dosage form will necessarily vary depending upon the host treated and the particular route of administration. For example, a formulation intended for oral administration to humans may contain the active agent compounded with an appropriate and convenient amount of excipients which may vary from about 5 to about 95 percent by weight of the total composition.

As another example, one embodiment of the present invention contemplates using and administering the calcium chelator, bisphosphonate, and/or citrate compounds together in a single dose that can be taken one or more times per day in order to inhibit the development of calcifications in vivo.

In order to use the formulation of calcium chelators, bisphosphonates, and/or citrate compounds for the therapeutic treatment (including prophylactic treatment) of mammals, including humans, the composition utilized may be formulated in accordance with standard pharmaceutical practice as a pharmaceutical composition as discussed above. According to this aspect of the invention there is provided a pharmaceutical and/or therapeutic composition of calcium chelators, bisphosphonates, and/or citrate compounds in association with a pharmaceutically acceptable diluent or carrier, wherein the calcium chelators, bisphosphonates, and/or citrate compounds are present in an amount for effectively treating or preventing pathological calcification, NB/CNP, heavy metal poisoning and calcification-induced diseases.

The composition of the present invention can be administered to a patient by any available and effective delivery system in dosage unit formulations containing conventional nontoxic pharmaceutically acceptable carriers, adjuvants, and vehicles as desired, such as a depot or a controlled release formulation.

2. Controlled/Extended/Sustained/Prolonged Release Administration:

One aspect of this invention provides methods of treating and/or preventing pathological calcification, heavy metal poisoning, NB/CNP and/or calcification-associated diseases by delivering the composition of the present invention to a patient as a controlled release formulation. As used herein, the terms “controlled” “extended” “sustained” or “prolonged” release of the composition of the present invention will collectively be referred to as “controlled release” and includes continuous or discontinuous, linear or non-linear release of the composition of the present invention.

There are many advantages for a controlled release formulation of the composition of the present invention. Among these are to effectively suppress calcification and Nanobacteria/Calcifying Nano-Particle growth during a period when the patient would not be readily able or willing to periodically ingest the composition of the present invention, the composition of the present invention is preferably administered following the evening meal and prior to bedtime in a single dose. The single dose of composition of the present invention preferably is administered via ingestion of one or more controlled release unit dosage forms, so that effective levels are maintained throughout an extended period of time.

A sample composition for a controlled release tablet may include, in admixture, about 5-30% high viscosity hydroxypropyl methyl cellulose, about 2-15% of a water-soluble pharmaceutical binder, about 2-20% of a hydrophobic component such as a waxy material, e.g., a fatty acid, and about 30-90% active ingredient.

More specifically, such a controlled release tablet may include: (a) about 5-20 percent by weight hydroxypropyl methylcellulose having a viscosity of about 10,000 CPS or greater, a substitution rate for the methoxyl group of about 7-30% and a substitution rate for the hydroxypropoxyl group of about 7-20%; (b) about 2-8 percent hydroxypropyl methylcellulose having a viscosity of less than about 100,000 CPS; methyl cellulose, or polyvinyl pyrollidone; (c) about 5-15 percent by weight hydrogenated vegetable oil or stearic acid; and (d) about 30-90% active ingredient.

High viscosity water-soluble 2-hydroxypropyl methyl cellulose (HPMC) is particularly preferred for use in the present tablets and in the controlled-release tablet coating, due to its sustaining properties with respect to release of the compositions of the present invention. A particularly preferred high viscosity HPMC has a nominal viscosity, two percent solution, of about 100,000 CPS, methoxyl content of about 19-24, a hydroxypropyl content of about 7-12 percent, and a particle size where at least 90% passes through a USS 100 mesh screen. (Methocel® K100MCR). Low viscosity HPMC is preferred as the binder component of the tablet. A particularly preferred low viscosity HPMC has a methoxyl content of about 20-30%, a hydroxylpropyl content of about 7-12 percent, and a particle size where 100% will pass through a USS No. 30 mesh screen and 99% will pass through a USS 40 mesh screen (Methocel® EIS). In some cases, a portion of the high viscosity HPMC can be replaced by a medium viscosity HPMC, i.e., of about 2000-8,000 cps.

The viscosities reported herein are measured in centipoises (cps or cP), as measured in a 2% by weight aqueous solution of the cellulose either at 20° C. using a rotational viscometer. A “high viscosity” cellulose ether possesses a viscosity of at least about 10,000 cps i.e., about 50,000-100,000 cps. A low-viscosity cellulose ether possesses a viscosity of less than about 100 cps, i.e., about 10-100 cps.

“Water soluble” for purposes of this application means that two grams of powdered cellulose ether can be dispersed by stirring into 100 grams of water at a temperature between 0° C.-100° C. to provide a substantially clear, stable aqueous composition or dispersion (when the dispersion is brought to 20° C.).

Useful hydrophobic components include natural and synthetic waxes such as beeswax, carnauba wax, paraffin, spermaceti, as well as synthetic waxes, hydrogenated vegetable oils, fatty acids, fatty alcohols and the like.

The controlled release tablets may be formulated to contain 0.1 to 3,000 mg of calcium chelator, bisphosphonate, and/or citrate compound, depending on the particular compositions used, and are ingested orally.

Preferably, these tablets will release about 10-35 wt-% of the total active ingredients of the present invention within about 2 hours in an in vitro dissolution test, and about 25-100 wt-% of the total active ingredients of the present invention in eight hours.

These controlled released tablets can also be coated so as to further prolong the release of the active ingredients of the present invention into the gastrointestinal tract, or to prevent its release into the stomach, in order to prevent or attenuate the gastrointestinal side effects which can accompany administration of calcium chelators such as EDTA.

For example, coatings comprising a major portion of a polymeric material having a high degree of swelling on contact with water or other aqueous liquids can be used to further prolong the release of the calcium chelators such as EDTA from the tablets core. Such polymers include, inter alia, cross-linked sodium carboxymethylcellulose (Acdisol-FMC), cross-linked hydroxypropylcellulose, hydroxymethylpropylcellulose, e.g., Methocel® K15M, Dow Chem. Co., carboxymethylamide, potassium methylacrylate divinylbenzene copolymer, polymethyl methacrylate, cross-linked polyvinylpyrrolidine, high molecular weight polyvinylalcohol, and the like. Hydroxypropylmethyl cellulose is available in a variety of molecular weights/viscosity grades from Dow Chemical Co. under the Methocel® designation. See also, Alderman (U.S. Pat. No. 4,704,285). These polymers may be dissolved in suitable volatile solvents, along with dyes, lubricants, flavorings and the like, and coated onto the prolonged release tablets, e.g., in amounts equal to 0.1-5% of the total tablet weight, by methods well known to the art. For example, see Remington's Pharmaceutical Sciences, A. Osol, ed., Mack Publishing Co., Easton, Pa. (16th ed. 1980) at pages 1585-1593.

Enteric coatings can also be provided to the prolonged release tablets to prevent release of the active ingredients of the present invention until the tablet reaches the intestinal tract. Such coatings comprise mixtures of fats and fatty acids, shellac and shellac derivatives and the cellulose acid phthlates, e.g., those having a free carboxyl consent of 9-15%. See, Remington's at page 1590, and Zeitova et al. (U.S. Pat. No. 4,432,966), for descriptions of suitable enteric coating compositions.

3. Transdermal Administration:

Transdermal delivery, involves delivery of a therapeutic agent through the skin for distribution within the body by circulation of the blood. Transdermal delivery can be compared to continuous, controlled intravenous delivery of a drug using the skin as a port of entry instead of an intravenous needle. The therapeutic agent passes through the outer layers of the skin, diffuses into the capillaries or tiny blood vessels in the skin and then is transported into the main circulatory system.

Also, transdermal patches may provide for the metering of the therapeutic composition to the area(s) of concern. For example, transdermal patches with the appropriate composition may be placed on the hands of a person suffering from arthritis wherein said composition is then metered directly into the area of concern. Another example may be the placement of a transdermal patch on a somatic lesion or other skin lesion for the local treatment of acute skin diseases as causes by calcification.

Transdermal patch devices which provide a controlled, continuous administration of a therapeutic agent through the skin are well known in the art. Such devices, for example, are disclosed in U.S. Pat. Nos. 4,627,429; 4,784,857; 5,662,925; 5,788,983; and 6,113,940, which are all incorporated herein by reference. Characteristically, these devices contain a drug impermeable backing layer which defines the outer surface of the device and a permeable skin attaching membrane, such as an adhesive layer, sealed to the barrier layer in such a way as to create a reservoir between them in which the therapeutic agent is placed. In one embodiment of the present invention a formulation of the composition of the present invention is introduced into the reservoir of a transdermal patch.

4. Opthamological Preparations or drops

The treatment of eye diseases involves using the composition comprising chelating agents, bisphosphonates, and/or citrate compounds as contained with a solution appropriate for the administration to the eye. Such a solution would contain certain pharmaceutically acceptable dilutants, carriers, humectants, emulsifiers, preservatives, and or desensitizers. Acceptable carriers include but are not limited to water, propylene glycol, polyethylene glycol, hydroxypropyl methyl cellulose, polyvinyl alcohol, carboxy methylcellulose, castor oil, carbomer, and light mineral. Emulsifiers include but are not limited to polysorbate 80 and tween 20. Preservatives include, but are not limited to benzalkonium chloride and sodium perborate.

Treatment or prevention using the composition containing one ore more of calcium chelators, bisphosphonates, and/or citrate compounds are helpful in the treatment of opthomalogical diseases both acute and chronic, localized or systemic, such as Band Keratopathy.

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A composition comprising at least one of calcium chelators, bisphosphonates, and/or citrate compounds.

2. The composition of claim 1, wherein said calcium chelator is comprising at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof.

3. The composition of claim 1, wherein said bisphosphonate is comprising at least one of alendronate, clodronate, ibandronate, incadronate, neridronate, palmidronate, risedronate, tiludronate, zoledronate, etidronate, oxidronate, and pharmaceutically acceptable salts thereof.

4. The composition of claim 1, wherein said citrate compound is comprising at least one of citrates, including sodium and potassium, magnesium citrate, phosphocitrate, and other complexes of citrate or organic or inorganic derivatives thereof.

5. A composition comprising at least one of calcium chelators, bisphosphonates, and/or citrate compounds, further comprising a pharmaceutically acceptable carrier, excipient or dilutant.

6. The composition of claim 5, in the form of a capsule, tablet, liquid or powder.

7. The composition of claim 5, in the form of a topical lotion, gel, or other preparation.

8. A method for treating or preventing the development of calcifications in vivo comprising administering a pharmaceutically effective amount of a composition comprising calcium chelators, bisphosphonates, and/or citrate compounds to a human or mammal.

9. A method for treating or preventing the growth of Nanobacterium/Calcifying Nano-Particles in vivo which comprises administering a pharmaceutically effective amount of a composition comprising calcium chelators, bisphosphonates, and pharmaceutical salts thereof.

10. A method for treating or preventing heavy metal poisoning which comprises administering a pharmaceutically effective amount of a composition comprising calcium chelators and bisphosphonates.

11. A composition comprising at least one of calcium chelators, bisphosphonates, and/or citrate compounds wherein said calcium chelator is selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof, and said bisphosphonate is selected from at least one of alendronate, clodronate, ibandronate, incadronate, neridronate, palmidronate, risedronate, tiludronate, zoledronate, etidronate, oxidronate, and pharmaceutically acceptable salts thereof, and said citrate compound is selected from at least one of citrate, including sodium and potassium salts, magnesium citrate, phosphocitrate and other complexes of citrate and or other organic and inorganic derivatives thereof, wherein said composition is further characterized by a combination of calcium chelator and bisphosphonate in a quantitative ratio from 100:1 to 0.01:1 by weight.

12. The composition of claim 11, wherein said composition is further characterized by a combination of calcium chelator and bisphosphonate in a quantitative ratio from 10:1 to 0.10:1 by weight.

13. The composition of claim 1 1, wherein said composition is further characterized by a combination of calcium chelator and bisphosphonate in a quantitative ratio from 3:1 to 0.33:1 by weight.

14. A method of treating and/or preventing calcification-associated diseases including, but not limited to heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Keratopathy, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Pyoderma gangrenosum, Dermatomyositis, eccrine sweat duct calcification, trichoepithelioma, pilomatrixoma, necrobiosis lipoidica, Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease in a patient, comprising delivering to said patient a composition comprising calcium chelators, bisphosphonates, and/or citrate compounds in an amount effective to reduce the occurance of and/or prevent calcification and calcification associated diseases including, but not limited to, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Keratopathy, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Pyoderma gangrenosum, Dermatomyositis, eccrine sweat duct calcification, trichoepithelioma, pilomatrixoma, necrobiosis lipoidica, Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease in an individual in need thereof.

15. The method of claim 14, wherein said composition is delivered to said patient as a controlled/sustained/extended/prolonged release composition.

16. The method of claim 14, wherein said composition is delivered to said patient as a controlled/sustained/extended/prolonged release composition, and wherein said controlled/sustained/extended/prolonged release composition comprises a thermoplastic polymer composition comprising a biocompatible polymer, a biocompatible solvent, calcium chelators, bisphosphonates, and/or citrate compounds and said controlled/sustained/extended/prolonged release composition is delivered to a bodily tissue or fluid in said patient, wherein the amounts of the polymer and the solvent are effective to form a biodegradable polymer matrix containing calcium chelators, bisphosphonates, and/or citrate compounds in situ when said composition contacts said bodily fluid tissue or fluid.

17. The method of claim 16, wherein said polymer is a poly(alkylene glycol) or a polysaccharide.

18. The method of claim 14, wherein said composition is delivered to said patient as a controlled/sustained/extended/prolonged release composition and wherein the composition further comprises a controlled/sustained/extended/prolonged release additive.

19. The method of claim 16, wherein said biocompatible polymer is selected from at least one of polylactides, polyglycolides, polyanhydrides, polyorthoesters, polycaprolactones, polyamides, polyurethanes, polyesteramides, polydioxanones, polyacetals, polyketals, polycarbonates, polyorthocarbonates, polyphosphazenes, polyhydroxybutyrates, polyhydroxyvalerates, polyalkylene oxalates, polyacrylates, polyalkylene succinates, poly(malic acid), poly(amino acids) and copolymers, terpolymers, cellulose diacetate, ethylene vinyl alcohol, startch acetate, hydroxyethyl starch, and copolymers and combinations thereof.

20. The method of claim 16, wherein said biodegradable polymer matrix releases calcium chelators, bisphosphonates, and/or citrate compounds by diffusion, erosion, or a combination of diffusion or erosion as the polymer matrix biodegrades in said patient.

21. The method of claim 16, wherein said calcium chelators, bisphosphonates, and/or citrate compounds are added to said polymer composition prior to administration such that said solid polymer matrix further contains said calcium chelators, bisphosphonates, and/or citrate compounds.

22. The method of claim 14, wherein said composition is delivered to said patient as a controlled/sustained/extended/prolonged release composition and wherein said controlled/sustained/extended/prolonged release is in tablet form.

23. A method of treating and/or preventing the development of calcifications in vivo comprising administering to a mammal a pharmaceutical composition in an amount that inhibits the growth of Nanobacteria/Calcifying Nano-Particles.

24. The method of claim 23, wherein the Nanobacteria/Calcifying Nano-Particles growth is inhibited by calcium chelators, bisphosphonates, and/or citrate compounds.

25. A method of treating, inhibiting and/or preventing the development of calcifications in vivo comprising administering to a mammal a pharmaceutical composition comprising calcium chelators, bisphosphonates, and/or citrate compounds.

26. The composition of claim 5, wherein said calcium chelator is selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof.

27. The composition of claim 5, wherein said bisphosphonate is selected from at least one of alendronate, clodronate, ibandronate, incadronate, neridronate, palmidronate, risedronate, tiludronate, zoledronate, etidronate, oxidronate, and pharmaceutically acceptable salts thereof.

28. The composition of claim 5, wherein said citrate compound is comprising at least one of citrate, including sodium and potassium salts, magnesium citrate, phosphocitrate and other complexes of citrate other organic and inorganic derivatives thereof.

29. The composition of claim 1, wherein said calcium chelators, bisphosphonates, and/or citrate compounds are administered in a daily dose within a range from 0.1 mg/day to 3,000 mg/day.

30. The composition of claim 1, wherein said calcium chelators, bisphosphonates, and/or citrate compounds are administered in a daily dose within a range from 10 mg/day to 2,000 mg/day.

31. The composition of claim 1, wherein said calcium chelators, bisphosphonates, and/or citrate compounds are administered in a daily dose within a range from 100 mg/day to 1,500 mg/day.

32. A controlled/sustained/extended/prolonged release preparation, comprising a pharmaceutically effective mixture of calcium chelators, bisphosphonates, and/or citrate compounds.

33. The composition of claim 32, wherein said calcium chelator is selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof.

34. The composition of claim 32, wherein said bisphosphonate is selected from at least one of alendronate, clodronate, ibandronate, incadronate, neridronate, palmidronate, risedronate, tiludronate, zoledronate, etidronate, oxidronate, and pharmaceutically acceptable salts thereof.

35. The composition of claim 32, wherein said citrate compound is selected from at least one of citrate, sodium or potassium salts, magnesium citrate, phosphocitrate and other complexes of citrate other organic and inorganic derivatives thereof.

36. A transdermal preparation designed to administer pharmaceutically effective amounts of calcium chelators, bisphosphonates, and/or citrate compounds into the blood stream.

37. The transdermal preparation of claim 36, wherein calcium chelators, bisphosphonates, and/or citrate compounds are present in a concentration sufficient that when applied to the skin a pharmaceutically effective steady state plasma concentration in the patient of said calcium chelators, bisphosphonates, and/or citrate compounds is produced.

38. A transdermal delivery system for application to the skin of a patient, comprising:
(a) a drug impermeable backing layer;
(b) an adhesive layer;
(c) a drug permeable membrane, wherein the membrane is positioned relative to the backing layer so as to form at least one drug reservoir compartment between the membrane and the backing layer; and
(d) a composition comprising calcium chelators, bisphosphonates, and/or citrate compounds contained within the drug reservoir compartment in a concentration sufficient such that the transdermal delivery system has an input rate when applied to the skin sufficient to produce a pharmaceutically effective steady state plasma concentration in the patient.

39. The method of claim 15, wherein said controlled/sustained/extended/prolonged release composition comprises applying a transdermal delivery system containing a mixture of calcium chelators, bisphosphonates, and/or citrate compounds to the skin of a patient and maintaining the transdermal delivery system in contact with the skin for a time sufficient to provide a pharmaceutically effective steady state plasma concentration in the patient.

40. The transdermal delivery system of claim 38, wherein said transdermal preparation is placed within close proximity to an area of caclific disease in order to be therapeutic to that area of disease.

41. The composition of claim 1, wherein said composition is in the form of a lotion, cream, gel, tincture, spray, or other form to be applied to the skin for the treatment of skin diseases characterized by calcification and/or Nanobacteria/Calcifying Nano-particles.

42. A composition of a topically applied preparation comprising calcium chelators, bisphosphonates, and/or citrate compounds to treat acute or chronic skin or dermatological diseases.

43. The composition of claim 42, wherein said calcium chelator is selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof; said bisphosphonate is selected from at least one of alendronate, clodronate, ibandronate, incadronate, neridronate, palmidronate, risedronate, tiludronate, zoledronate, etidronate, oxidronate, and pharmaceutically acceptable salts thereof; and said citrate compound is selected from at least one of citrate, including sodium and potassium salts, magnesium citrate, phosphocitrate and other complexes of citrate other organic and inorganic derivatives thereof.

44. The composition of claim 42, wherein said topically applied preparation is placed within close proximity to an area of caclific disease in order to be therapeutic to that area of disease.

45. A method for treating and/or preventing the growth of Nanobacteria/Calcifying Nano-particles in vivo which comprises administering a pharmaceutically/therapeutically effective amount of a composition to a patient comprising bisphosphonates.

46. The method of claim 45, wherein said bisphosphonate is selected from at least one of alendronate, clodronate, ibandronate, incadronate, neridronate, palmidronate, risedronate, tiludronate, zoledronate, etidronate, oxidronate, and pharmaceutically acceptable salts thereof.

47. The method of claim 45, wherein said pharmaceutically effective bisphosphonate is administered in a dosage of 0.1 to 3000 mg/day.

48. The method of claim 45, wherein said pharmaceutically effective bisphosphonate is administered in a dosage of 10 to 2000 mg.day.

49. The method of claim 45, wherein said pharmaceutically effective bisphosphonate is administered in a dosage of 100 to 1500 mg.day.

50. The method of claim 45, wherein said pharmaceutically effective bisphosphonate is delivered to said patient as a controlled/sustained/extended/prolonged release composition.

51. A controlled/sustained/extended/prolonged release preparation comprising a pharmaceutically active bisphosphonate.

52. The composition of claim 51, wherein said bisphosphonate is selected from at least one of alendronate, clodronate, ibandronate, incadronate, neridronate, palmidronate, risedronate, tiludronate, zoledronate, etidronate, oxidronate, and pharmaceutically acceptable salts thereof.

53. The composition of claim 14, wherein said composition is in the form of eye drops for the treatment of Band Keratopathy or similar diseases of the eye as associated with pathological calcification.

54. The composition of claim 53, wherein said composition contains appropriate pharmaceutical additives to be delivered in the form of eye drops.

55. A composition comprising calcium chelators, bisphosphonates, and/or citrate compounds for the treatment of Band Keratopathy or diseases of the eye caused by pathological calcification.

56. The composition of claim 55, wherein said calcium chelator is selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof; said bisphosphonate is selected from at least one of alendronate, clodronate, ibandronate, incadronate, neridronate, palmidronate, risedronate, tiludronate, zoledronate, etidronate, oxidronate, and pharmaceutically acceptable salts thereof; and said citrate compound is selected from sodium and potassium salts, magnesium citrate, phosphocitrate and other complexes of citrate other organic and inorganic derivatives thereof.

57. The composition of claim 55, wherein said composition is administrered in the form eye drops for the treatment of calcific diseases of the eye.

58. A composition comprising bisphosphonates and/or citrate compounds for the treatment or prevention of kidney stones as caused by nephrolithiasis.

59. The composition of claim 58, wherein said bisphosphonates is selected from at least one of alendronate, clodronate, ibandronate, incadronate, neridronate, palmidronate, risedronate, tiludronate, zoledronate, etidronate, oxidronate, and pharmaceutically acceptable salts thereof; and said citrate compound is selected from sodium and potassium salts, magnesium citrate, phosphocitrate and other complexes of citrate other organic and inorganic derivatives thereof.

60. The composition of claim 58, wherein said bisphosphonates and/or citrate compounds further comprises pharmaceutically acceptable carriers, excipients, or diluents.

61. The composition of claim 60, in the form of a capsule, tablet, liquid, or powder.

62. A method of administering a therapeutic composition comprising bisphosphonates and/or citrate compounds to a patient to treat and/or prevent the formation of kidney stones as caused by nephrolithiasis.

63. The method of claim 62, wherein said bisphosphonates is selected from at least one of alendronate, clodronate, ibandronate, incadronate, neridronate, palmidronate, risedronate, tiludronate, zoledronate, etidronate, oxidronate, and pharmaceutically acceptable salts thereof; and said citrate compound is selected from sodium and potassium salts, magnesium citrate, phosphocitrate and other complexes of citrate other organic and inorganic derivatives thereof.

64. The method of claim 62, wherein said composition is delivered to a patient as an oral dosage such as capsule, pill, or tablet.

65. The method of claim 62, wherein said composition is delivered to said patient as a controlled/sustained/extended/prolonged release composition.
